(19) United States
(12) Patent Application Publication
(10) Pub. No.: US 2022/0168740 A1
(43) Pub. Date: Jun. 2, 2022

(54) SYSTEMS, KITS, AND RELATED METHODS OF MAKING FECAL TRANSPLANTS

(71) Applicants: University of Washington, Seattle, WA (US); Seattle Children's Hospital d/b/a Seattle Children's Research Institute, Seattle, WA (US)

(72) Inventors: Thomas Lendvay, Seattle, WA (US); William DePaolo, Seattle, WA (US); Blake Hannaford, Seattle, WA (US); Scott Weissman, Seattle, WA (US); David Suskind, Seattle, WA (US)

(73) Assignees: University of Washington, Seattle, WA (US); Seattle Children's Hospital d/b/a Seattle Children's Research Institute, Seattle, WA (US)

(21) Appl. No.: 17/441,186

(22) PCT Filed: Mar. 20, 2020
(86) PCT No.: PCT/US2020/023766

§ 371 Date: Sep. 20, 2021

Related U.S. Application Data
(60) Provisional application No. 62/822,702, filed on Mar. 22, 2019.

(51) Int. Cl. B01L 3/00; A61K 9/48; A61K 35/74

(52) U.S. Cl. CPC B01L 3/502761; A61K 9/48; B01L 2300/0681; B01L 3/502753; B01L 2200/0647; A61K 35/74

(57) ABSTRACT

Systems, kits, and related methods of making fecal transplants are described. In an embodiment, the system comprises a sample collector defining a sample collection chamber shaped to collect a fecal sample from fecal matter; a sample processing module defining a sample port shaped to receive the sample collector and comprising: a channel positioned to receive at a proximal end of the channel the fecal sample from the sample collection chamber when the sample collector is received by the sample port; and a capsule fluidically coupled to a distal end of the channel adapted to receive a portion of the fecal sample passed therethrough; and a sample processing unit comprising: a housing defining an opening shaped to receive the sample processing module; and an actuator disposed in the housing and configured to urge the sample from the sample collection chamber through the channel to the capsule.

### CROSS-REFERENCE TO RELATED APPLICATIONS

This application claims the benefit of U.S. Provisional Application No. 62/822,702, filed on Mar. 22, 2019, the contents of which are incorporated herein by reference in their entirety.

### BACKGROUND

A healthy intestinal microbiome has a normal balance of bacteria. There is a rapidly growing body of research supporting the importance of each individual's healthy gut microbiome, established over an individual's lifetime. Antibiotics and chemotherapy can damage this balance with even a single dose. The disruption of this complex ecosystem can affect the immune system and the way food is processed in the body. Furthermore, disrupted intestinal bacteria balance has been linked to mental health disorders, obesity, malnutrition, and inflammatory diseases.

Currently, patients do not have an accessible, reliable way to re-establish their healthy microbiome when it becomes imbalanced (dysbiosis) and many patients are using probiotics that may not yield any benefit or, even worse, could delay the return of healthy gut bacteria. In this regard, it may be desirable to ingest one's own fecal matter in order to establish a healthy biome. For fecal matter to be put into a usable form from original raw form, the fecal matter conventionally must be collected and sent to an organization that processes the fecal matter and cannot be processed at the point-of-use. This conventional process takes days and may decrease the value of consumption of the fecal material. Current fecal transplant solutions also rely on a few donors making it impossible or difficult to scale the fecal matter.

Accordingly, there is a need for devices, kits, and methods for producing fecal transplants without the need for a centralized laboratory, such as in a point-of-use setting.

### SUMMARY

The present disclosure provides systems, kits, and related methods for producing fecal transplant capsules to address this need and provide other related advantages. In various aspects, the present disclosure provides solutions to the noted problems of conventional fecal transplant by providing a user with systems and kits to be used at the point-of-use by allowing the user to collect fecal matter, insert the fecal matter into the system/kit, in certain embodiments mix and/or filter the fecal matter, place the fecal matter into a usable form, such as in a capsule, for use at the point-of-use and/or storage and use at a future time.

Accordingly, in an aspect, the present disclosure provides a system for producing fecal transplant capsules. In an embodiment, the system comprises a sample collector defining a sample collection chamber shaped to collect a fecal sample from fecal matter; a sample processing module defining a sample port shaped to receive the sample collector and comprising: a channel positioned to receive at a proximal end of the channel the fecal sample from the sample collection chamber when the sample collector is received by the sample port; and a capsule fluidically coupled to a distal end of the channel adapted to receive a portion of the fecal sample passed therethrough; and a sample processing unit comprising: a housing defining an opening shaped to receive the sample processing module; and an actuator disposed in the housing and configured to urge the sample from the sample collection chamber through the channel to the capsule.

In another aspect, the present disclosure provides a kit for producing fecal transplant capsules. In an embodiment, the kit comprises a sample collector defining a sample collection chamber shaped to collect a fecal sample from fecal matter; and a sample processing module defining a sample port shaped to receive the sample collector and comprising: a channel positioned to receive at a proximal end of the channel the fecal sample from the sample collection chamber when the sample collector is received by the sample port; and a capsule fluidically coupled to a distal end of the channel adapted to receive a portion of the fecal sample passed therethrough.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the claimed subject matter will become more readily appreciated as the same become better understood by reference to the following detailed description, when taken in conjunction with the accompanying drawings, wherein:

FIG. 1 is a perspective view of a system, in accordance with an embodiment of the disclosure;

FIG. 2A is a cross section view of a sample processing module, in accordance with an embodiment of the disclosure;

FIG. 2B is a cross section view of the sample processing module of

FIG. 2A showing a fecal sample received by a sample port of the sample processing module, in accordance with an embodiment of the disclosure.

FIG. 2C is a cross section view of the sample processing module of FIG. 2A showing the fecal sample entering a mixer of the sample processing module, in accordance with an embodiment of the disclosure;

FIG. 2D is a cross section view of the sample processing module of FIG. 2A showing the fecal sample passing through the mixer, in accordance with an embodiment of the disclosure;

FIG. 2E is a cross section view of the sample processing module of FIG. 2A showing the fecal sample entering a filter of the sample processing module, in accordance with an embodiment of the disclosure;

FIG. 2F is a cross section view of the sample processing module of FIG. 2A showing a filtered fecal sample exiting the filter, in accordance with an embodiment of the disclosure;

FIG. 2G is a cross section view of the sample processing module of

FIG. 2A showing the fecal sample filling capsules of the sample processing module, in accordance with an embodiment of the disclosure;

FIG. 2H is a cross section view of the sample processing module of FIG. 2A showing the filled capsules removed from the sample processing module and coupled to a pull tab, in accordance with an embodiment of the disclosure;

FIG. 3 is a cross section view of a sample processing module in accordance with an embodiment of the disclosure;

FIG. 4A is a perspective view of a sample collector, in accordance with an embodiment of the disclosure;

FIG. 4B is a perspective view of a system including the sample collector of FIG. 4A, in accordance with an embodiment of the disclosure; and

FIG. 4C is a cross section view of the system of FIG. 4B, in accordance with an embodiment of the disclosure.

### DETAILED DESCRIPTION

Embodiments of systems, kits, and related methods for making fecal transplant capsules are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment.

Furthermore, the particular features, structures, or characteristics may be combined in any suitable manner in one or more embodiments.

In an aspect, the present disclosure provides a system for producing fecal transplant capsules. As described in further detail below, the systems of the present disclosure are suitable to prepare capsules and/or other forms of a fecal transplant comprising a portion of a fecal sample. Such capsules comprising the portion of the fecal sample may be consumed by a patient for a fecal transplant, such as an autologous fecal transplant following a course of antibiotics, chemotherapy, or other medical procedure that depletes or decreases gut microbiota.

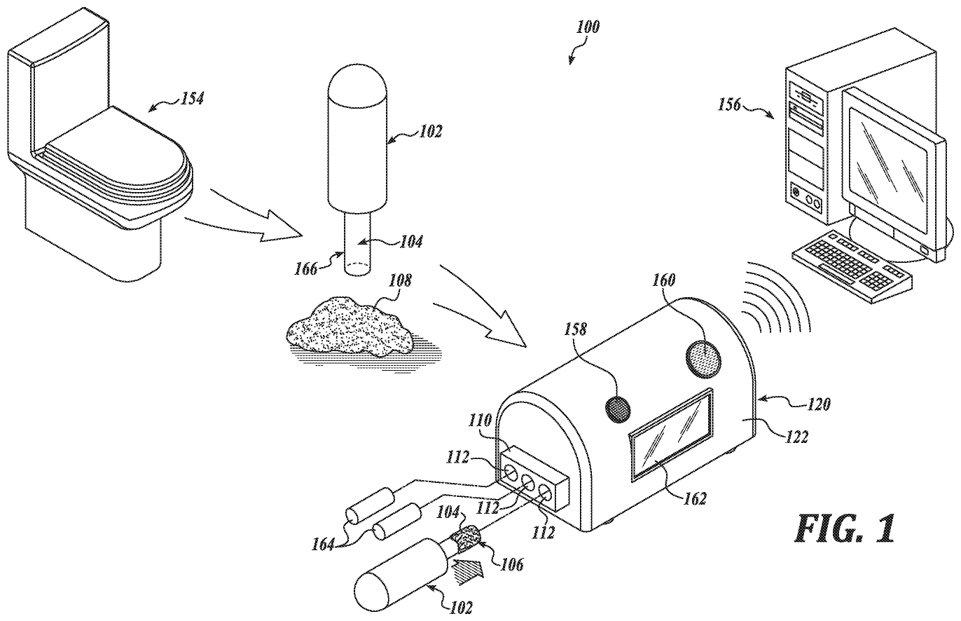

In this regard, attention is directed to FIG. 1, in which a perspective view of a system 100, in accordance with an embodiment of the disclosure, is illustrated. As shown, the system 100 includes a sample collector 102 shaped to collect a fecal sample 106 from fecal matter 108, a sample processing module 110 including sample ports 112 shaped to receive the sample collector 102, and a sample processing unit 120 shaped to receive the sample processing module 110.

In an embodiment, the sample collector 102 defines a sample collection chamber 104 shaped to collect the fecal sample 106 from the fecal matter 108. In an embodiment the sample collection chamber 104 is shaped to collect a sample core from the fecal matter 108. As shown, the sample collector 102 is a punch, such as a disposable punch, including a cylindrical member 166 defining the sample collection chamber 104. While a cylindrical sample collection chamber 104 is described, it will be understood that other shapes are possible and within the scope of the present disclosure. In an embodiment, the sample collector 102 is used in or adjacent to a toilet 154, and in this regard, the system 100 is suitable for at the point of use.

As above, the sample processing module 110 includes a sample port 112 shaped to receive the sample collector 102. In the illustrated embodiment, the sample processing module 110 defines a number of sample ports 112 each shaped to receive the sample collector 102. As will be described in greater detail further herein, such sample ports 112 are configured to receive a number of fecal samples or portions of a single fecal sample 106. In an embodiment, the system 100 includes plugs 164 shaped to be received by the sample ports 112 thereby sealing the sample ports 112.

In use, the sample collector 102 is received by the sample processing module 110, such as by inserting the cylindrical member 166 into one of the sample ports 112. In this regard, a user may expel the fecal sample 106 from the sample collector 102 into the sample processing module 110 for processing therein. Likewise, in use, a user can place the plugs 164 in the sample ports 112 not receiving a sample collector 102, thereby preventing backflow of the fecal sample 106 out of the unused sample ports 112.

As shown, the sample processing module 110 is received and carried by a housing 122 of the sample processing unit 120. In this regard and as discussed further herein, the sample processing unit 120 is configured to move the fecal sample 106 through the sample processing module 110 to process the fecal sample 106, such as by mixing, filtering, and/or disposing the fecal sample 106 in capsules. In an embodiment, however, the sample processing unit 120 is fluidically isolated from both the sample collector 102, such as a sample collection chamber 104 of the sample collector 102, and an interior portion of the sample processing module 110. In this regard, portions of the system 100 that come in contact with the fecal sample 106, such as the sample collector 102 and the sample processing module 110, may be disposable, whereas other portions of the system 100, such as the sample processing unit 120 may be reused, such as in receiving unused sample processing modules for processing additional fecal samples therein.

In an embodiment, the sample processing unit 120 is shaped be disposed on a desktop or tabletop. In an embodiment, the sample processing unit 120 is shaped to be portable, such as shaped and sized to be carried by a single person. In this regard, the sample processing unit 120 is suitable for use and storage in a laboratory, hospital, or other setting where fecal samples are typically processed and in other non-conventional locations and settings, such as in point-of-use settings.

In the illustrated embodiment, the sample processing unit 120 is shown to include a microphone 158, such as for receiving voice commands from a user, and a speaker 160, such as for communicating information about operation of the system 100 and results of fecal sample 106 encapsulation. The sample processing unit 120 is also shown to include a display 162, such as for displaying results or status of the system 100 to a user.

As shown, the sample processing unit 120 is in operative communication with a computer 156, shown here as a desktop computer 156, such as to exchange signals therebetween. Such operative communication is suitable to provide an indication of whether and when processing of a fecal sample 106 is complete or if an error has occurred. While wireless communication is shown, such as through WiFi, Bluetooth, and the like, other configuration are possible such as wired communication. In another embodiment, the sample processing unit 120 itself is configured to provide such signals.

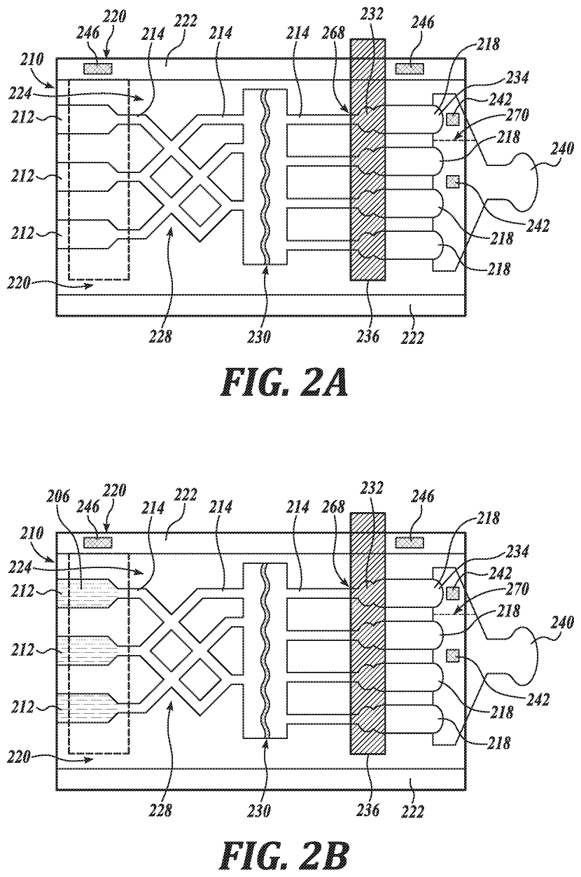

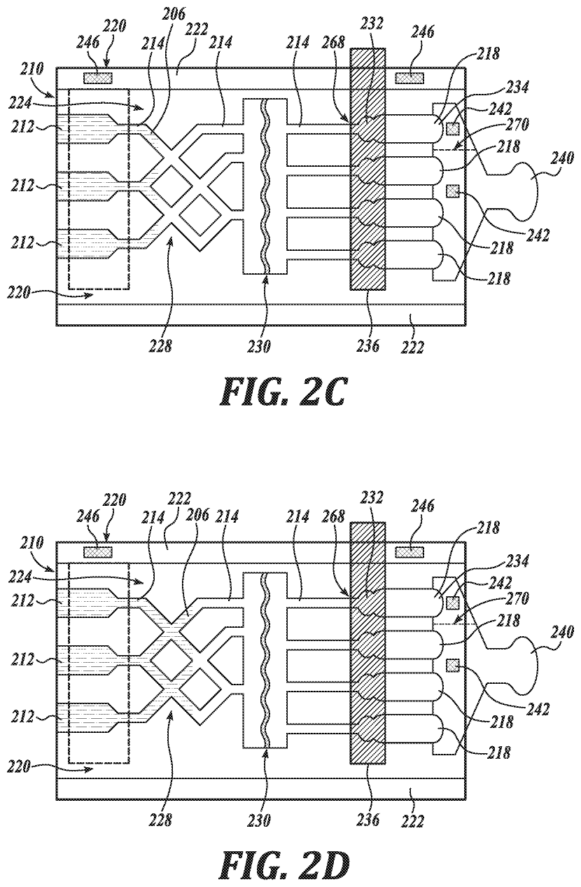

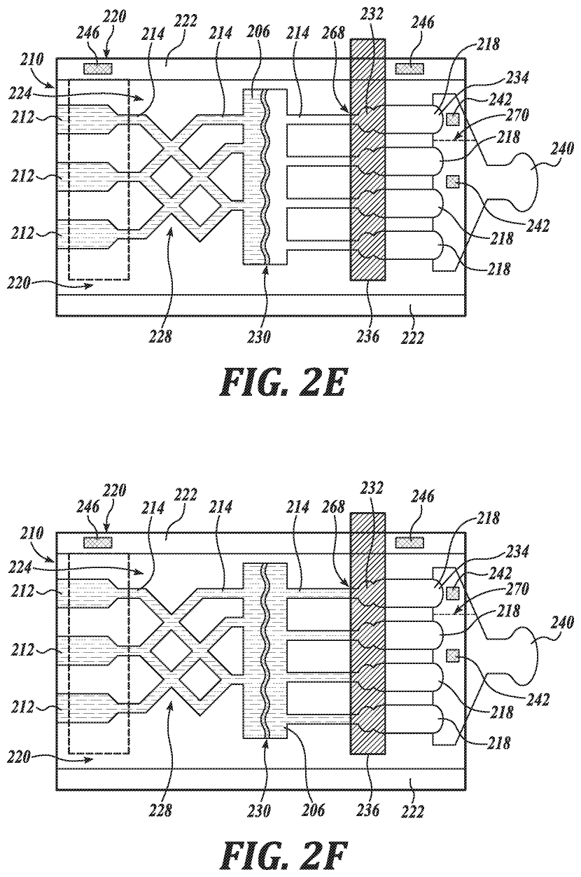

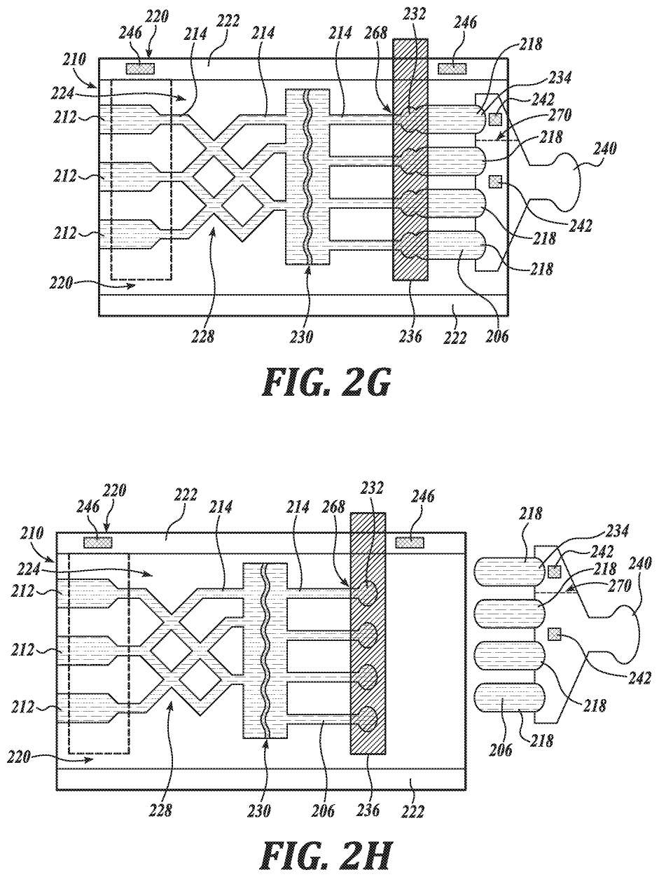

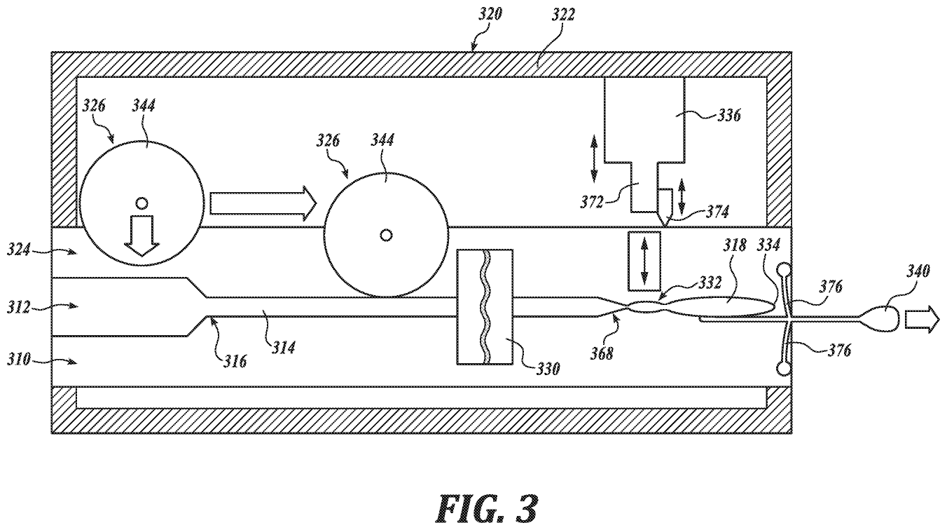

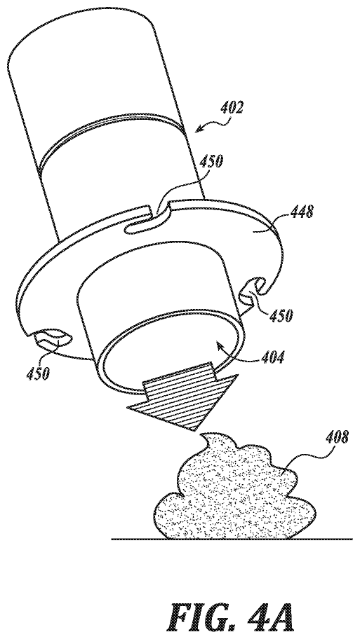

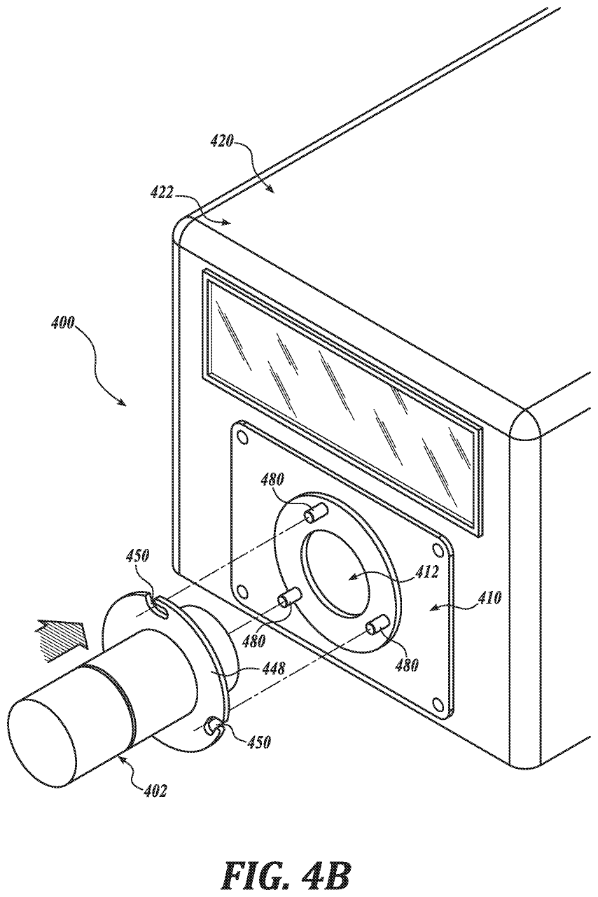

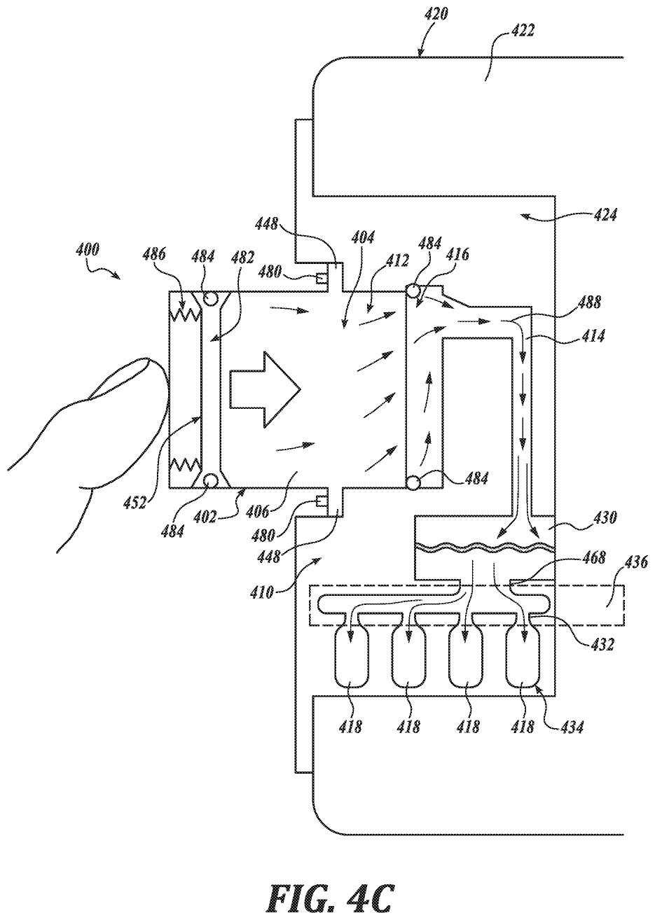

In an embodiment, the sample processing module defines a channel in fluidic communication with the sample port and the sample capsules. In that regard, attention is directed to FIGS. 2A-2H in a which a cross section view of a sample processing module 210, in accordance with an embodiment of the disclosure, is illustrated. As shown, the sample processing module 210 includes a number of sample ports 212; a channel 214 positioned to receive at a proximal end 216 of the channel 214 the fecal sample 206 from the sample collection chamber of a sample collector (see, e.g., FIG. 1) when a sample collector is received by the sample port 212; and a capsule 218 fluidically coupled to a distal end 268 of the channel 214 adapted to receive a portion of the fecal sample 206 passed therethrough. As shown, the capsules 218 define a first end 232, shown here as an open end 232, fluidically coupled to the distal end 268 of the channel and a closed end 234 opposite the open end 232. In use, the fecal sample 206 is transported through the sample ports 212 and into the channel 214 (see FIGS. 2B and 2C), such as through actuation of an actuator in the sample processing unit 220 (see, e.g., FIG. 3) or actuation of a piston in the sample collector (see, e.g., FIGS. 4A-4C). In an embodiment, “fluidic communication” refers to two or more components in which a fluid disposed in one component is configured to contact or travel to another of the two or more components, such as by fluid flow therebetween. In an embodiment, “fluidic communication” includes configurations in which the two or more components are not immediately adjacent to one another. In another embodiment, “fluidic communication” includes configurations in which the two or more components are not perfectly sealed.

In the illustrated embodiment, the sample processing module 210 is shown received by sample processing unit 220. As discussed further herein such as with respect to FIG. 1, the sample processing module 210 is shaped to be received by the sample processing unit 220, such as received by opening 224 defined by housing 222 of sample processing unit 220, and may be releasably coupled thereto. In this regard, in an embodiment, the sample processing module 210 is separate and separable from the sample processing unit 220.

As shown, the sample processing module 210 includes a mixer 228. In the illustrated embodiment, mixer 228 is in fluidic communication with the channel 214 and the sample port 212. The mixer 228 is configured to mix the fecal sample 206 as it passes through the mixer 228. Such mixing is advantageous where, for example, the fecal sample 206 will be disposed in a number of capsules 218. By homogenizing or otherwise mixing the fecal sample 206, the portions of the fecal sample 206 in each of the multiple capsules 218 are more evenly representative of the fecal sample 206 as a whole. In certain instances, one or more of the capsules 218 are tested to determine, for example, a number of bacteria in the capsule 218. By having a homogenized portion of the fecal sample 206 in the capsules 218, such a tested capsule 218 is suitable to provide a result more representative of the encapsulated fecal sample 206 portions in untested capsules 218.

As shown in FIGS. 2C and 2D, the mixer 228 includes a labyrinth of interconnected paths in fluidic communication with the sample ports 212. As the fecal sample 206 moves through the labyrinth of interconnected paths, the fecal sample(s) 206 is/are mixed and homogenized. In an embodiment, the mixer 228 is optional. While a labyrinth of interconnected paths is illustrated, it will be understood that other mixers are within the scope of the present disclosure, such as a magnetic stirrer disposed in a chamber, which is moved by a motor disposed in the sample processing unit 220.

The sample processing module 210 is shown to further include a filter 230. In the illustrated embodiment, the filter 230 is shown in fluidic communication with the channel 214 and downstream from the mixer 228. The filter 230 is configured to filter the fecal sample 206 as it passes through the filter 230. See, for example, FIGS. 2E and 2F. In this regard, the filter 230 is configured to remove, for example, portions of the fecal sample 206 having a relatively large cross section, which may not include bacteria useful in fecal transplant. In an embodiment, the filter 230 is optional. In an embodiment, the filter 230 includes a wire mesh defining a number of apertures configured to allow a portion of the fecal sample 206 to pass therethrough.

As shown, the sample processing module 210 defines a number of channels 214 in fluidic communication at a proximal end 216 with the filter 230 and, at a distal end 268, with the capsules 218. As the filtered fecal sample 206 passes from the filter 230, it is configured to be received by the capsules 218, as shown in FIGS. 2F and 2G.

In an embodiment, the capsules 218 are rigid and, in an initial state, evacuated to allow fecal sample 206 to fill an interior void of the capsules 218 without a change in capsule volume. In an embodiment, the capsules 218 are initially collapsed (see, e.g., FIG. 3) and flexible and are, thus configured to be filled and expand as the fecal sample 206 enters an interior volume of the capsules 218. In an embodiment, the capsules 218 include an outer sleeve and an inner sleeve coaxially disposed within the outer sleeve, wherein an interior volume of the inner sleeve is in fluidic communication with the channel 214 to receive the fecal sample 206.

In an embodiment, the capsules 218 are composed of a material, such as a cellulose-based material, which dissolves inside the human gastrointestinal tract in order to properly dispose the fecal sample 206 inside a patient's body, such as within the patient's gut to repopulate the patient's intestinal microbiome.

In an embodiment, the capsules 218 are sized and shaped to be swallowed by a patient. In an embodiment, the capsules 218 are sized and shaped as a suppository. In an embodiment, the sample processing module 210 and the sample processing unit 220 are configured to provide a fecal sample solution, such as by diluting the fecal sample 206 with a sterile saline solution, the fecal sample solution comprising a portion of the fecal matter for use as an enema.

As shown, not all of the fecal sample 206 is disposed in the capsules 218 after the fecal sample 206 is processed. In this regard, a portion of the fecal sample 206 is disposed within the channel 215, mixer 228, the filter 230, and the like. While some portion of the fecal sample 206 remains in portions of the sample processing module 210, the sample processing module 210 is configured to dispose a sufficient portion of the fecal sample 206 in the capsules 218 to fill the capsules 218 to provide a sufficient dose for an effective fecal transplant. In an embodiment, the capsules 218 are configured to hold about one ounce of the fecal sample 206, whereas a total volume of the fecal sample is in a range of about 2 ounces to about 4 ounces.

In an embodiment, the capsules 218 are coupled to a pull tab 240, as shown in FIG. 2H. In use, the pull tab 240 is pulled to separate the capsules 218 from the channel 214 and other portions of the sample processing module 210. In this regard, the capsules 218 now containing the processed portions of the fecal sample 206 are physically coupled together but separated from sample processing module 210.

In the illustrated embodiment, the pull tab 240 includes an identifier 242 associated the capsules 218 and fecal sample 206 disposed therein. Such an identifier 242 can be used to track the capsules 218, the fecal sample 206 contained therein, and a fecal sample donor. In an embodiment, the identifier 242 is selected from a barcode, an RFID tag, a serial number, human-readable numbering and coding, dot patterns, serial numbers, DNA sequence information, handwritten text entry fields, and combinations thereof. In an embodiment, the identifier 242 comprises a 1D, 2D, or other barcode, which contains a hash code or a very large random number. In an embodiment, the bar code is printed on the pull tab 240 by the sample processing unit 220. In an embodiment, the barcode is pre-printed with a unique hash code or very large random number during its manufacture. Once read or printed by the sample processing unit 220, this identifying information can be transmitted by the sample processing unit 220 to one or more of a central server, a local server, a cloud computing instance, and a mobile device. The identifying information from the identifier 242 on the pull tab 240 can be associated with the identity of the user, the date, time and location of sample collection, and information from sensors 246 inside the sample processing unit 220 indicating the quality, quantity, and adequacy of sample as it passes through the sample processing module 210 and sensors 246 deployed by the sample processing unit 220 in contact or proximity with the sample processing module 210.

In another embodiment, the removable pull tab 240 is divided into two or more sections, such as by perforations 270, each of which has a barcode or other identifier 242. In an embodiment, at least one of the sections is configured to be easily detached by a user from the user for shipping to a central lab. The remaining sections can be frozen and stored at the collection site. Shipment to the central lab can be accomplished with dry ice or using conventional methods used for shipping samples for the fecal occult blood test. In this regard, the central lab can conduct any analysis of the fecal sample 206, and results can be matched to the fecal samples retained by the user via the matching identifiers 242.

In the illustrated embodiment, the sample processing unit 220 includes a housing 222 defining an opening 224 shaped to receive the sample processing module 210. As shown, portions of sample processing module 210 are shaped to come in contact with a fecal sample 206, such as the sample port 212 and the channel 214 are fluidically isolated from the sample processing unit 220. In this regard, the sample processing unit 220 is not in fluidic communication with channel 214 or the sample collection chamber 204 when the sample processing module 210 is received by the sample processing unit 220 and the sample collector 202 is received by the sample port 212. Likewise, in an embodiment, sample processing unit 220 surfaces internal to the sample processing module 210 and in fluid communication with the fecal sample 206 are removeable/replaceable.

may be used repeatedly to receive several sample processing modules for processing of several fecal samples, such as without cross contamination of the several fecal samples.

In an embodiment, the sample processing unit 220 includes one or a number of sensors 246 positioned to generate a signal based on a presence or absence of fecal sample 206 within the sample processing module 210. In this regard, the sensors 246 are configured to determine whether the fecal sample 206 has been received by and moved through the sample processing module 210. In an embodiment, the sensor is configured to generate a signal based on a presence or absence of the fecal sample 206 in one or more of the sample port 212, the channel 214, the capsule 218, and combinations thereof. In an embodiment, such signals are used to choreograph operation of an actuator, a sealing mechanism 236, the cutter, etc. to move the fecal sample 206 from the sample port 212 to the capsules 218 and separate the capsules 218 from other portions of the sample processing module 210. Likewise, in an embodiment, the signal is used to generate an alert if the sample processing unit 220 and/or sample processing module 210 is working improperly, such as if the filter 230 is clogged or if the fecal sample 206 is not disposed in the capsule 218.

In an embodiment, the sensors 246 are configured to intermittently, continuously, or otherwise measure variable quantities in the sample processing module 210 during loading and processing of the fecal sample 206. In an embodiment, such quantities include, but are not limited to, pressure, temperature, pH, optical transmission, reflection, and scattering properties of the fecal sample 206. In an embodiment, such sensors 246, thus, include without limitation an optical density sensor, a conductivity sensor, a spectral light sensor, and combinations thereof. In one embodiment, a color sensor is integrated within the sample processing unit 220 including one or several Light-Emitting Diodes (LED) which are configured to emit an associated color or colors (optical wavelength range) and photodetector responsive to the wavelengths of the associated color(s). In an embodiment, other sensors 246 include a MOSFET, chemically sensitive field-effect transistor (ChemFET), a sensor in which a gate of the device is modified to attract constituent molecules in the fecal sample 206, which then modifies its electrical response in proportion to the targeted constituent molecule.

Sensed quantities may include a degree to which the fecal sample 206 has flowed into channels 214, passages, cavities, filters, and capsules 218 in the sample processing module 210. For example, in one embodiment, a camera is positioned to image the entire sample processing module 210 or a portion of the sample processing module 210, which may be made of transparent material, and determine which channels, chambers, capsules 218, ports, and other portions of the sample processing module 210 contain sample and which portions are empty. In this and other embodiments, sensor information may be used to alter the duration or magnitude or direction of actuation such that fecal sample 206 is moved through the sample processing module 210. Sensors 246 and actuators in the sample processing unit 220 may also comprise feedback loops in which actuators affect a physical variable or characteristic of part of the sample processing module 210, and sensors 246 measure such a variable or characteristic and the actuator output is thereby adjusted to regulate or control such a variable or characteristic quantity inside the sample processing module 210.

In some embodiments, sensors 246 are disposed entirely inside the sample processing unit 220, entirely inside the sample processing module 210, or some sensors 246 are disposed in the sample processing unit 220 while other sensors 246 may be disposed in the sample processing module 210. In some embodiments, multiple sensors 246, disposed in the same or in differing parts of the sample processing unit 220, are configured to measure the same quantity in order to increase confidence in the measurement, and/or to measure a difference in the material as it moves through stages of the sample processing module 210. In an embodiment, some sensors 246 are configured to generate signals which do not pertain to the fecal sample 206 at all, such as based on a degree to which the sample processing module 210 is properly inserted into the sample processing unit 220; the ambient temperature, humidity, air pressure of the location; or identification information from the sample processing module 210.

In an embodiment, the sample processing unit includes an actuator disposed in the housing and configured to urge the sample from the sample collection chamber through the channel to the capsule. In that regard, attention is directed to FIG. 3 in which a sample processing module 310 is received by a sample processing unit 320, in accordance with an embodiment of the disclosure, is illustrated.

As shown, the sample processing module 310 defines a sample port 312 shaped to receive a sample collector (see, e.g., FIG. 1) and comprises a channel 314 positioned to receive at a proximal end 316 of the channel 314 the fecal sample from the sample collection chamber when the sample collector is received by the sample port 312; and a capsule 318 fluidically coupled to a distal end 368 of the channel 314 adapted to receive a portion of the fecal sample passed therethrough. The sample processing unit 320 is shown to include a housing 322 defining an opening 324 shaped to receive the sample processing module 310; and an actuator 326 disposed in the housing 322 and configured to urge the sample from the sample collection chamber through the channel 314 to the capsule 318.

As above, the capsule 318 is in fluidic communication with the channel 314 and, when present in the channel 314, the fecal sample. In an embodiment, the capsule 318 defines first end 332, shown here as an open end 332, fluidically coupled to the distal end 368 of the channel 314 and a closed end 334 opposite the open end 332. In order to remove the capsule 318 from the sample processing module 310, the sample processing unit 320 can further comprise a sealing mechanism 336 shaped to seal the open end 332 of the capsule 318. Such a sealing mechanism 336 can comprise a heat source 372 configured to melt or otherwise seal a portion of the capsule 318 adjacent to the open end 332. In an embodiment, the sealing mechanism 336 includes a source of ultrasound 372 configured to seal the portion of the capsule 318 adjacent to the open end 332. In an embodiment, the sealing mechanism 336 further comprises a cutter, such as one or more blades 374, a laser, etc., configured to separate the channel 314 from the sealed capsule(s) 318.

In the illustrated embodiment, the actuator 326 includes a compressive element 344 configured to compress the channel 314 to urge the fecal sample therethrough by peristalsis. In an embodiment, in use the compressive element 344 presses down on the channel 314 and moves laterally as illustrated by the arrows to move the fecal sample through the channel 314, such as from a proximal portion of the channel 314 adjacent to the sample port 312 to a distal portion of the channel 314 abutting the filter 330. In an embodiment, the compressive element 344 includes a roller shaped to compress the channel 314 and roll along a surface thereof to urge the fecal sample through the channel 314. In an embodiment, the channel 314 is compliant and flexible such that it compresses in response to pressure from the compressive element 344, thereby displacing fecal sample disposed in the channel 314.

While rollers are illustrated, it will be understood that other configurations of the actuator 326 are possible, such as a row of pistons configured to sequentially compress a portion of the channel 314 to provide a peristaltic wave within the channel 314. Likewise, in an embodiment, the actuator 326 operates to provide pneumatic, magnetic, or hydraulic pressure to the fecal sample within the sample processing module 310.

In an embodiment, the sample processing module 310 includes one or more capsules 318 coupled to a pull tab 340. As discussed further herein with respect to

FIGS. 2A-2H, the pull tab 340 is configured to be selectively removed from other portions of the sample processing module 310, such as by a user. In an embodiment, the capsule(s) 318 may exit the sample processing module 310 via flaps 376 or doors which open as the capsule(s) 318 are pulled out. In an embodiment, such flaps 376 are configured to prevent any future use of the sample processing module 310 by breaking away from the sample processing module 310 when the capsule(s) 318 are pulled out. Alternatively, the doors may be hinged and configured to reclose and be latched. In an alternative embodiment, an opening in the sample processing unit 320, on a side opposite to the entry point of the sample processing module 310 is configured to allow the user to pull out the pull tab 340 without first removing the sample processing module 310 from the desktop sample processing unit 320. After removal of the capsules 318 by the user, the sample processing module 310 may then be removed from the sample processing unit 320 and thrown away, recycled, or cleaned and manufactured.

In an embodiment, a fecal sample is moved through the system of the present disclosure through a user-powered actuation. In this regard, attention is directed to FIGS. 4A-4C in which a system 400, in accordance with an embodiment of the disclosure is illustrated. FIG. 4A is a perspective view of a sample collector 402, in accordance with an embodiment of the disclosure. FIG. 4B is a perspective view of a system 400 including the sample collector 402. FIG. 4C is a cross section view of the system 400, in accordance with an embodiment of the disclosure.

As shown, the system 400 includes a sample collector 402 defining a sample collection chamber 404 shaped to collect a fecal sample 406 from fecal matter 408; a sample processing module 410 defining a sample port 412 shaped to receive the sample collector 402 and comprising: a channel 414 positioned to receive at a proximal end 416 of the channel 414 the fecal sample 406 from the sample collection chamber 404 when the sample collector 402 is received by the sample port 412; and a capsule 418 fluidically coupled to a distal end 468 of the channel 414 adapted to receive a portion of the fecal sample 406 passed therethrough; and a sample processing unit 420 comprising a housing 422 defining an opening 424 shaped to receive the sample processing module 410. As shown, the capsules 418 define an open end 432 fluidically coupled to the distal end 468 of the channel 414 and a closed end 434 opposite the open end 432 and the sample processing module 410 further includes a sealing mechanism 436 configured to seal the capsules 418 from the channel 414, such as after a portion of the fecal sample 406 is disposed in the capsules 418.

In use, the sample collector 402 can be pressed down onto fecal matter 408 such that the sample collection chamber 404 captures a portion of the fecal matter 408, such as a sample core. In the illustrated embodiment, the sample collector 402 is a cylinder defining an interior lumen, which defines at least in part the sample collection chamber 404. While a cylinder is shown, it will be understood that other shapes and configurations are possible within the scope of the present disclosure.

In the illustrated embodiment, the sample collector 402 defines a flange 448 encircling the sample collection chamber 404. The flange 448 is shaped to abut the sample processing module 410 when the sample collection chamber 404 is received by the sample port 412. See, e.g., FIG. 4B. The sample collector 402 is also shown to include a locking mechanism 450 shaped to cooperatively couple with the sample processing module 410. As shown, the locking mechanism 450 includes a number of slots defined by the flange 448. The slots are shaped to cooperatively couple with the sample processing module 410, in particular posts 480 protruding from the sample processing module 410 adjacent to the sample port 412, to seal a coupling between the sample collection chamber 404 and the sample port 412. In this regard, the sample collector 402 is shaped to be securely coupled to the sample processing module 410 ensuring that the fecal sample 406 is less likely to escape from the sample collection chamber 404 or the sample port 412. While posts 480 and slots 450 are illustrated, it will be understood that other cooperative coupling structures between the sample collector 402 and sample processing module 410 are possible, such as threads, locking grooves, Luer locks, and the like.

As shown in FIG. 4C, the sample collector 402 comprises a piston 482 slideably disposed in the sample collection chamber 404. Through actuation of the piston 482, the piston 482 is configured to expel the fecal sample 406 from the sample collection chamber 404. As shown, such actuation is configured to move the fecal sample 406 through the channel 414, the filter 430, and into the capsules 418. Thus, in the illustrated embodiment, actuation of the piston 482 provides a force sufficient to process the fecal sample 406 through the sample processing module 410.

In an embodiment, an actuation portion 452 of the sample collector 402, shown here as a portion configured to receive an actuation force from a user, is not in fluidic communication with the sample collection chamber 404. The sample collector 402 is shown to further include an accordion folded membrane 486 disposed between the piston 482 and the sample collection chamber 404 to prevent or limit contamination of a user's, for example, finger with the fecal sample 406. In this regard, a user can actuate or depress the actuation portion 452 of the piston 482, such as with a finger, thumb, palm, or the like, without contaminating, for example, a portion of their hand. In an embodiment, the sample collector 402 includes a seal 484 encircling the piston 482 configured to fluidically isolate the actuation portion 452 from the sample collection chamber 404.

In the illustrated embodiment, the sample processing module 410 includes a seal 484, such as a gasket, o-ring, or other sealing member, positioned to seal a coupling between the sample collector 402 and the sample processing module 410 when the sample collector 402 is received by the sample port 412. In this regard, fecal sample 406 is transmitted from the sample collection chamber 404 into the channel 414.

As shown, the channel 414 defines a convergence zone 488 between the seal 484 and the filter 430, which functions to pre-condition the fecal sample 406. Such conditioning may be suitable to condition the fecal sample 406 for processing in the filter 430 and in the mixing the fecal sample 406.

In another aspect, the present disclosure provides kits for producing fecal transplant capsules. In an embodiment, the kits of the present disclosure comprise a sample collector and a sample processing module. In an embodiment, the sample collector and sample processing module are the sample collectors and sample processing modules discussed further herein with respect to FIGS. 1, 2A-2H, 3, and 4A-4C, in any combination. In an embodiment, one or more of the components of the kits are disposable.

In an embodiment, the sample collector defines a sample collection chamber shaped to collect a fecal sample from fecal matter. In an embodiment, the sample collector defines a flange shaped to abut the sample processing module when the sample collector is received by a sample port of the sample processing module, and the sample collector comprises a locking mechanism shaped to cooperatively couple with the sample processing module to seal a coupling between the sample collection chamber and the sample por. See, for example, FIGS. 4A-4C.

In an embodiment, the sample collector comprises a piston slideably disposed in the sample collection chamber and configured to expel the fecal sample from the sample collection chamber through actuation of the piston, the sample collector further comprises a seal disposed between the piston and the sample collection chamber and configured to seal an actuation portion of the sample collector from the sample collection chamber. See, for example, FIGS. 4A-4C.

In an embodiment, the sample processing module defines a sample port shaped to receive the sample collector and comprising: a channel positioned to receive at a proximal end of the channel the fecal sample from the sample collection chamber when the sample collector is received by the sample port; and a capsule fluidically coupled to a distal end of the channel adapted to receive a portion of the fecal sample passed therethrough. In an embodiment, the capsule defines a first end, such as an open end, fluidically coupled to the distal end of the channel and a closed end opposite the open end. See, for example, FIGS. 2A-2H.

In an embodiment, the capsule is a first capsule, and wherein the sample processing module further comprises a second capsule in fluidic communication with the distal end of the channel to receive a second portion of the fecal sample. See, for example, FIGS. 2A-2H. In an embodiment, the first capsule and the second capsule are disposed on a pull tab releaseably coupled to the channel. See, for example, FIGS. 2A-2H.

Certain processes explained above are described in terms of computer software and hardware. The techniques described may constitute machine-executable instructions embodied within a tangible or non-transitory machine (e.g., computer) readable storage medium, that when executed by a machine will cause the machine to perform the operations described.

The order in which some or all of the steps are described in each process should not be deemed limiting. Rather, one of ordinary skill in the art having the benefit of the present disclosure will understand that some of the steps may be executed in a variety of orders not illustrated, or even in parallel.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.). Additionally, the processes may be embodied within hardware, such as an application specific integrated circuit (“ASIC”) or otherwise.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

While illustrative embodiments have been illustrated and described, it will be appreciated that various changes can be made therein without departing from the spirit and scope of the invention.

1. A system for producing fecal transplant capsules, the system comprising:
a sample collector defining a sample collection chamber shaped to collect a fecal sample from fecal matter;
a sample processing module defining a sample port shaped to receive the sample collector and comprising:
a channel positioned to receive at a proximal end of the channel the fecal sample from the sample collection chamber when the sample collector is received by the sample port; and
a capsule fluidically coupled to a distal end of the channel adapted to receive a portion of the fecal sample passed therethrough; and
a sample processing unit comprising:
a housing defining an opening shaped to receive the sample processing module; and
an actuator disposed in the housing and configured to urge the sample from the sample collection chamber through the channel to the capsule.

2. The system of claim 1, wherein the sample processing module comprises a mixer in fluidic communication with the channel configured to mix the fecal sample as the fecal sample passes through the mixer.

3. The system of claim 1, further comprising a filter in fluidic communication with the channel and configured to filter the fecal sample as the fecal sample passes through the filter.

4. The system of claim 1, wherein the capsule defines an open end fluidically coupled to the distal end of the channel and a closed end opposite the open end, the sample processing unit further comprising a sealing mechanism shaped to seal the open end of the capsule.

5. The system of claim 1, wherein the capsule is a first capsule, and wherein the sample processing module further comprises a second capsule in fluidic communication with the distal end of the channel to receive a second portion of the fecal sample passed therethrough.

6. The system of claim 5, wherein the first capsule and the second capsule are disposed on a pull tab releaseably coupled to the channel.

7. The system of claim 6, wherein the pull tab comprises an identifier associated with the first capsule and the second capsule.

8. The system of claim 1, wherein the actuator comprises one or more compressive element configured to compress the channel to urge the fecal sample therethrough by peristalsis.

9. The system of claim 1, wherein the sample processing unit further comprises a sensor configured to generate a signal based on a presence or absence of the sample in one or more of the sample port, the channel, and the capsule.

10. The system of claim 1, wherein the sample processing unit is not in fluidic communication with channel or the sample collection chamber when the sample processing module is received by the sample processing unit and the sample collector is received by the sample port.

11. The system of claim 1, wherein the sample collector defines a flange encircling the sample collection chamber and shaped to abut the sample processing module when the sample collection chamber is received by the sample port.

12. The system of claim 1, wherein the sample collector comprises a locking mechanism shaped to cooperatively couple with the sample processing module to seal a coupling between the sample collection chamber and the sample port.

13. The system of claim 1, wherein the sample collector comprises a piston slideably disposed in the sample collection chamber and configured to expel the fecal sample from the sample collection chamber through actuation of the piston.

14. The system of claim 13, wherein an actuation portion of the sample collector is not in fluidic communication with the sample collection chamber.

15. A kit for producing fecal transplant capsules, the kit comprising:
a sample collector defining a sample collection chamber shaped to collect a fecal sample from fecal matter; and
a sample processing module defining a sample port shaped to receive the sample collector and comprising:
a channel positioned to receive at a proximal end of the channel the fecal sample from the sample collection chamber when the sample collector is received by the sample port; and
a capsule fluidically coupled to a distal end of the channel adapted to receive a portion of the fecal sample passed therethrough.

16. The kit of claim 15, wherein the capsule defines an open end fluidically coupled to the distal end of the channel and a closed end opposite the open end.

17. The kit of claim 15, wherein the capsule is a first capsule, and wherein the sample processing module further comprises a second capsule in fluidic communication with the distal end of the channel to receive a second portion of the fecal sample.

18. The kit of claim 17, wherein the first capsule and the second capsule are disposed on a pull tab releaseably coupled to the channel.

19. The kit of claim 15, wherein the sample collector defines a flange shaped to abut the sample processing module when the sample collector is received by the sample port, and wherein the sample collector comprises a locking mechanism shaped to cooperatively couple with the sample processing module to seal a coupling between the sample collection chamber and the sample port.

20. The kit of claim 15, wherein the sample collector comprises a piston slideably disposed in the sample collection chamber and configured to expel the fecal sample from the sample collection chamber through actuation of the piston, and wherein the sample collector further comprises a seal disposed between the piston and the sample collection chamber and configured to seal an actuation portion of the sample collector from the sample collection chamber.
